(19) United States
(12) Patent Application Publication
(10) Pub. No.: US 2026/0306370 A1
(43) Pub. Date: Oct. 8, 2026

(54) O/W EMULSION CONTAINING ETHANOL

(71) Applicant: DR. KURT WOLFF GMBH & CO. KG, Bielefeld (DE)

(72) Inventors: Erik SCHULZE ZUR WIESCHE, Bielefeld (DE); Petra BÖPPLE, Bielefeld (DE); Sabrina SCHEIDELER, Bielefeld (DE)

(73) Assignee: DR. KURT WOLFF GMBH & CO. KG, Bielefeld (DE)

(21) Appl. No.: 19/699,879

(22) Filed: Jun. 5, 2026

Related U.S. Application Data
(62) Division of application No. 17/918,532, filed on Apr. 15, 2021.

(30) Foreign Application Priority Data
Apr. 15, 2020 (EP) 20169713.3

(51) Int. Cl. A61K 8/06; A61K 8/34; A61K 8/37; A61Q 19/10

(52) U.S. Cl. CPC A61K 8/062; A61K 8/34; A61K 8/37; A61Q 19/10

(57) ABSTRACT

The invention relates to an oil-in-water emulsion containing 15.0 to 30.0 wt.-% ethanol.

### CROSS-REFERENCE TO RELATED APPLICATIONS

This application is a divisional of U.S. National Stage application Ser. No. 17/918,532, filed Oct. 12, 2022, which is a U.S. National Stage Application filed under 35 U.S.C. § 371 of International Application No. PCT/EP2021/059820, filed Apr. 15, 2021, which claims the benefit of European Patent Application No. 20169713.3, filed Apr. 15, 2020. All of these applications are hereby incorporated by reference in their entirety.

### FIELD OF THE INVENTION

The present invention is directed to an O/W emulsion comprising 15.0 to 30.0 wt.-% ethanol.

### BACKGROUND OF THE INVENTION

Disinfecting products of the prior art are commonly sold as solutions, ointments or gels containing more than 62 wt.-% ethanol. Gels are the most common sanitizing/disinfecting products. These gels have good disinfecting properties but can have skin-irritating or corrosive properties, in particular when applied multiple times a day (as is mandatory for health care professionals). Thus, the prior art disinfectants can leave the skin feeling irritated, rashed, dry and brittle.

Furthermore, such disinfectants can ultimately lead to unwanted side-effects for people with sensitive skin conditions, such as patients suffering from e.g. neurodermitis. The skin barrier may thus be weakened and applying disinfectant can have adverse effects. Instead of protecting the skin from external bacterial and viral pathogens, the skin barrier is weakened to such an extent that the use of a disinfectant may even promote infections eventually. Therefore, the disinfectants that are commercially available are unsuitable for people with skin sensitive conditions.

In addition, aqueous alcohol solutions and gels quickly evaporate on the skin, thus minimizing the contact-time of the ethanol with the skin and therefore leading to non-optimal disinfection. Even more, quick evaporation of the alcohol can lead to nausea and headaches when inhaled after prolonged or multiple exposure due to several applications per day.

Additionally, the prior art disinfectants containing ethanol are in particular unsuitable for people suffering from alcohol addiction and/or humans with decreased levels of the enzyme ADH (alcohol dehydrogenase) due to the quick evaporation and adsorption of the alcohol by inhalation.

Furthermore, the disinfectant compositions of the prior art are flammable having a flash point of around 17° C. Therefore, special care needs to be taken during manufacturing, transport and storage of these disinfectants.

In view of the above problems and disadvantages, it was an object of the present invention to provide an improved disinfecting composition that overcomes the insufficiencies of the prior art.

### SUMMARY OF THE INVENTION

These objects have surprisingly been solved by an oil-in-water emulsion of the present invention that comprises
(a) an oil phase;
(b) an aqueous phase; and
(c) an emulsifier,
   wherein the emulsion further comprises 15.0 to 30.0 wt.-% ethanol.

It has surprisingly been found according to the present invention that the oil-in-water emulsion both maintains high effectiveness against pathogens, in particular against bacterial infections and enveloped virus infections such as infections with SARS-COV-2, while at the same time maintains the skin barrier function and is mild, stable, non-irritant, has moisturizing properties and provides a smooth, comfortable and clean feeling. A combined effect in terms of both high effectiveness against pathogens and improvement of skin barrier function was totally unexpected. In addition, it was surprisingly found that the composition leads to a prolonged contact of the skin with the ethanol and increased evaporation time of the ethanol.

In particular, it has surprisingly been found that according to the above aspect, the disinfecting properties can be maintained while at the same time the negative side effects of the available disinfecting compositions are decreased. In addition, it has surprisingly been found that the ethanol forms a stable and homogenous composition in the oil-in-water emulsion i.e. the formation of multiple distinct phases is not observed even after prolonged storage.

In another aspect, the present invention relates to the use of the oil-in-water emulsion of the present invention for use in the prevention of a bacterial, mycotic, or enveloped viral disease.

In another aspect, the present invention relates to the non-therapeutic use of the oil-in-water emulsion of the present invention as a cosmetic, preferably for topical application to the skin, in particular for combined skin moisturizing and skin disinfection.

### DETAILED DESCRIPTION

The following definitions are relevant in connection with the embodiments of the present invention.

The meaning of the term “comprising” is to be interpreted as encompassing all the specifically mentioned features as well optional, additional, unspecified ones, whereas the term “consisting of” only includes those features as specified. Therefore, “comprising” includes as a limiting case the composition specified by “consisting of”.

The term “weight-%” or “wt.-%” is to be understood to refer to the weight amount of the component relative to the total weight amount of the respective composition, if not specified otherwise.

Preferred embodiments according to the invention are defined hereinafter. These embodiments are preferred, even if not denoted as such. The preferred embodiments are preferred alone or in combination. Further, it is to be understood that the following preferred embodiments refer to all aspects of the present invention, i.e. oil-in-water emulsion, the oil-in-water emulsion for use in the prevention of a bacterial or enveloped viral disease, and the non-therapeutic use.

In an embodiment, the invention relates to an oil-in-water emulsion comprising
(a) an oil phase;
(b) an aqueous phase; and
(c) an emulsifier,
   wherein the emulsion further comprises 15.0 to 30.0 wt.-% ethanol.

In a preferred embodiment, the emulsion comprises 15.0 to 25.0, 17.0 to 23.0 or 19.0 to 21.0 wt.-% ethanol.

In an embodiment, the oil-in-water emulsion (in the following sometimes also referred to as O/W emulsion) further comprises 1.0 to 10.0 wt.-% of cetearyl ethylhexanoate, preferably 2.0 to 8.0 wt.-% or 4.0 to 7.0 wt.-%, as a skin care component. It has surprisingly been found that in particular in the presence of cetearyl ethylhexanoate, a composition can be provided that results in a smooth and comfortable feeling of the skin while at the same time providing a stable composition.

In an embodiment, the emulsifier is present in an amount of 1.0 to 15.0 wt.-%.

In an embodiment, the emulsifier is selected from the group of fatty alcohol esters, glycerol fatty acid esters, polyoxyethylene ethers of one or more fatty alcohols, polyglycerol ethers of fatty alcohols, polyglycerol esters of fatty acids, and mixtures thereof.

The term “fatty alcohol esters” refers to C₂-C₂₀ allkanoates of C₆-C₂₈ branched or linear alkyls or alkenyls, wherein the prefix “Cₓ-C_{y}” denotes the number of carbon atoms. It is preferred that the C₂-C₂₀ alkanoate is acetate. Non-limiting examples of fatty alcohol esters include n-octylacetate and lauryl acetate. These compounds can additionally act as fragrance in the oil-in-water composition.

In the present application the term “glycerol fatty acid ester” refers to a glycerol mono or di fatty acid ester, i.e. a compound composed of a molecule of glycerol linked to a single fatty acid via an ester bond. Examples are glycerol monostearate, glycerol monobehenate, glycerol monocaprylate, glycerol monocaprate and glycerol monolaurate.

Preferably, the polyoxyethylene ethers of one or more fatty alcohols is selected from the group of steareth-2, steareth-21, macrogol cetostearyl ether 12, ceteareth-25, macrogol cetostearyl ether 20 and mixtures of the aforementioned compounds. More preferably, the polyoxyethylene ethers of one or more fatty alcohols is selected from the group of ceteareth-25, macrogol cetostearyl ether 20 and mixtures of the aforementioned compounds. In the current composition ceteareth-25, macrogol cetostearyl ether 12 and macrogol cetostearyl ether 20 promote the dispersion of the oil phase in the aqueous of the emulsion. They have ideal dispersion properties due to the lengths of the respective alcohol residues.

The term “polyglycerol ethers of fatty alcohols” refers to a compound of the formula R¹O—(C₃H₆O₂)ₙ—H, wherein R¹ is a C₆-C₂₈ branched or linear alkyl or alkenyl group and n is an integer greater than 1, preferably an integer from 2 to 10. It is preferred that the oil-in water emulsion comprises 1.0 to 8.0 wt.-% of polyglycerol ethers of fatty alcohols, and/or 1.0 to 5.0 wt.-% of glycerol fatty acid esters, and/or 1.0 to 5.0 wt.-% of polyoxyethylene ethers of one or more fatty alcohols.

The term “polyglycerol esters of fatty acids” refers to compounds containing a polyglycerol unit as well as at least one C₆-C₂₆ alkyl or alkenyl carboxylic acid unit. These two units may be combined directly by an ester bond or contain a linking unit that links these two units together. Non-limiting example of this group are polyglyceryl-3 methylglucose distearate, polyglycerol ester of interesterified ricinoleic acid, polyglycerol polycrinoleate, polyglyceryl dimerate isostearate, polyglyceryl polyricinoleate (Admul WOL 1403), polyglyceryl-1 dipolyhydroxystearate (Dehymuls PGPH), polyglyceryl-2-laurate, polyglyceryl-2 sesquiisostearate, polyglyceryl-3 beeswax (Cera Bellina), polyglyceryl-3 cetyl ether (Chimexane NL), polyglyceryl-3 distearate (Cremophor GS 32), polyglyceryl-3 oleate, polyglyceryl-3 methyl glycose distearate, polyglyceryl-4-caprate (polyglycerol caprate T2010190), polyglyceryl-4 diisostearate/polyhydroxystearate/sebacate (Isolan GPS), and polyglyceryl-4 isostearate.

In yet another embodiment, the oil-in-water emulsion has a dynamic viscosity of more than 2500 mPa's at 20° C., preferably a dynamic viscosity of 4500 to 14000 mPa·s at 20° C. The dynamic viscosity can be measured according to ISO 2555:2018 using a Brookfield DVII+viscometer.

In an embodiment, the oil-in-water emulsion comprises the oil phase in an amount of 0.1 to 20.0 wt.-%, wherein the oil phase comprises fatty alcohols, alkylcarbonates, alkylesters, waxes, natural oils, or mixtures thereof.

The term “fatty alcohols” encompasses C₆-C₂₈ alcohol, preferably C₁₂-C₂₀ alcohols, wherein the prefix “Cₓ-C_{y}” denotes the number of carbon atoms. The alcohol may be a linear or branched alcohol. Non-limiting examples include lauryl alcohol, myristyl alcohol, cetyl alcohol, stearyl alcohol, and ceryl alcohol. It is in particular preferred that the fatty alcohol is a mixture of C₁₆ and C₁₈ fatty alcohol called cetearyl alcohol.

The term “alkylcarbonate” refers to difunctionalized carbonate alkyl esters. It is preferred that the alkyl groups are identical, but the alkyl groups can be selected independently. The alkyl group may be any C₃-C₂₀ branched, linear or cyclic alkyl. It is preferred that the alkyl group is a C₃-C₂₀ linear alkyl group, even more preferably a capryl group.

The term “wax” refers to natural waxes, such as jojoba wax, and synthetic waxes such as polyglycerin-3.

Non-limiting examples of natural oils comprise canola oil, soy oil, sunflower oil, almond oil, grape oil, avocado oil, castor oil, glycine, jojoba oil, arnica oil, hibiscus oil, licorice oil and oils from Areca catechu, Crocus sativus, Curcuma longa, Glycyrrhiza glabra, green tea, Crataevea murula, Rosmarinus Officinalis, buckwheat seeds, Emblica officinale, Ginkgo biloba, Centella asiatica, Psoraliea corylifolia, Citrus limonum, Aloe Vera, matricaria recutita, Thea viridis, Vitis vinifera, Daucus carota, Lycopersicon esculentum, Allium sativum and Hamamelis virginiana. The natural oil may have various useful properties and can i.a. be used as anti-inflammatory, anti-irritant, antimicrobial or fragrance component.

The oil-in-water emulsion according to the current invention can include further excipients which are known to the skilled person. These excipients are preferably cosmetically, pharmaceutically and/or dermatologically acceptable compounds. For instance, the further excipients are selected from the group of pH-modifiers, buffers, detergents, solubilizers, humectants, fillers, bioadhesives, emollients, preservatives, bactericides, surfactants, perfumes, thickeners (also referred to as thickening agents hereinafter), softening agents, moisturizing agents, oils, fats, polyols, and polymers. Suitable excipients are described in “Cosmetic Formulation of Skin Care Products”, Draelos et al., Taylor & Francis Group, 2006.

In an embodiment, the oil-in-water emulsion further comprises a thickening agent, wherein the thickening agent is preferably selected from xanthan gum, polyacrylic acid, cellulose ethers, and mixtures thereof. It is preferred that thickening agent is present in an amount of 0.01 to 5 wt.-%, 0.05 to 3 wt.-% or 0.1 to 0.8 wt.-%. Non-limiting examples of the above cellulose ethers contain methylcellulose, hydroxypropyl cellulose, hydroxyethyl cellulose, and hydroxyethylmethyl cellulose.

In an embodiment, the oil-in-water emulsion further comprises 0.01 to 5.0 wt.-% of an emollient, wherein the emollient is preferably dimethicone and/or cyclopentasiloxane. Emollients are beneficial for skin lubricity and are soothing. In some embodiments, the oil-in-water emulsion comprises humectant agents, such as glycerin, hyaluronic acid, ammonium lactate, and panthenol.

In an embodiment, the weight ratio (b)/(a) is from 2.0 to 15.0, preferably from 3 to 10. This weight ratio ensures a stable emulsion in the presence of ethanol that does not separate into distinct phases.

In an embodiment, the oil-in-water emulsion further comprises a vitamin. Non-limiting examples of vitamins include niacin, biotin, vitamin E (also referred to as tocopherol), vitamin A, vitamin C, and dexpanthenol. Vitamins are antioxidants and can have various additional useful properties for maintaining a healthy skin barrier.

In an embodiment, the oil-in-water emulsion comprises 40.0 to 80.0 wt.-% water phase, 0.1 to 20 wt.-% oil phase, 15.0 to 30.0 wt.-% ethanol, 1.0 to 15.0 wt.-% of an emulsifier, 1.0 to 10.0 wt.-% of cetearyl ethylhexanoate, and 0.01 to 3 wt.-% of a thickening agent.

In a preferred embodiment, the oil-in-water emulsion comprises 40.0 to 80.0 wt.-% water phase, 0.1 to 20 wt.-% oil phase, 15.0 to 30.0 wt.-% ethanol, 1.0 to 15.0 wt.-% of an emulsifier, 1.0 to 10.0 wt.-% of cetearyl ethylhexanoate, 0.01 to 3 wt.-% of a thickening agent, 0.01 to 2.0 wt.-% of an emollient, and 0.01 to 1.0 wt.-% of a vitamin.

In an embodiment, the invention relates to the above oil-in-water emulsion for use in the prevention of a bacterial, mycotic, or enveloped viral disease.

In an embodiment, the oil-in-water emulsion is topically applied to the skin, preferably to the hand. In a preferred embodiment, the oil-in-water emulsion is applied according to DIN EN 1500:2017 for at least 10 seconds, at least 30 seconds, at least 60 seconds, or at least 120 seconds.

In an embodiment, the disease is an infection with an enveloped virus belonging to the group consisting of Poxviridae, Paramyxoviridae, Filoviridae, Orthomyxoviridae, Astroviridae, and Coronaviridae, or wherein the disease is an infection with a bacterium belonging to the group of Staphylococcus aureus, Enterococcus hirae, Pseudomonas aeruginosa, Escherichia coli, Haemophilus influenza, and Streptococcus pneumoniae. It is particularly preferred that the disease is an infection with an influenza virus. It is also particularly preferred that the disease is a bacterial infection with Staphylococcus aureus, Enterococcus hirae, Escherichia coli or Pseudomonas aeruginosa.

In an embodiment, the disease is an infection with a virus belonging to the family of Coronaviridae, preferably an infection with SARS-COV-2.

In an embodiment, the invention relates to the non-therapeutic use of the oil-in-water emulsion as a cosmetic, preferably for topical application to the skin, in particular for combined skin moisturizing and skin disinfection.

### EXAMPLES

### Example 1: Skin Tolerability

An oil-in-water emulsion containing 61.175 wt.-% water, 20.00 wt.-% ethanol, 6.00 wt.-% cetearyl ethylhexanoate, 2.50 wt.-% polyglyceryl-3 methylglucose distearate, 2.00 wt.-% glyceryl stearate, 2.00 wt.-% dicapryl carbonate, 2.00 wt.-% panthenol, 1.50 wt.-% cetearyl alcohol, 0.5 wt.-% carbomer, 0.4 wt.-% polyglycerin-3, and 0.3 wt.-% xanthan gum was provided as Test Example 1.

A pre-determined amount of 2 mL of said oil-in-water emulsion was topically applied to the hand of ten test subjects three times a day over the course of 14 days. The skin tolerability was subsequently measured, wherein
“x” denotes no change in the skin properties of the test subjects;
“n” denotes the occurrence of mild rashes, irritations or dryness; and
“o” denotes the occurrence of a more moist and smoother skin.

After 14 days, all test subjects denoted “o” or “x” and none of the test subjects denoted “n”.

### Example 2: In Vitro Anti-Bacterial Efficacy

The oil-in-water emulsion of Test Example 1 was tested undiluted in accordance with DIN EN ISO 1276 against S. aureus, E. coli, E. hirae, P. aeruginosa.

As Comparative Example 1, a composition comprising water, glycerin, cetearyl alcohol, cetearyl ethylhexanoate, isohexadecane, sorbitol, ethanol, butyrospermum parkii (shea) butter, dimethicone, sodium cetearyl sulfate, panthenol, tocopheryl acetate, allantoin, rosa centifolia flower extract, serine, glycine, urea, glucose, citric acid, sodium citrate, lactic acid, sodium lactate, phenoxyethanol, benzyl alcohol, parfum, hexyl cinnamal, linalool, butylphenyl methylpropional, alpha-isomethyl ionone, and citronellol (EUBOS Sensitive Hand Repair & Schutz) was tested in the same manner as Example 1. Comparative Example 1 contains less than 10 wt.-% ethanol.

As Comparative Example 2, an oil-in-water emulsion containing 81.175 wt.-% water, 6.00 wt.-% cetearyl ethylhexanoate, 2.50 wt.-% polyglyceryl-3 methylglucose distearate, 2.00 wt.-% glyceryl stearate, 2.00 wt.-% dicapryl carbonate, 2.00 wt.-% panthenol, 1.50 wt.-% cetearyl alcohol, 0.5 wt.-% carbomer, 0.4 wt.-% polyglycerin-3, and 0.3 wt.-% xanthan gum was tested in the same manner as Example 1. Comparative Example 2 does not contain any ethanol.

The exposure time was 300 (600) seconds. It was found that the oil-in-water emulsions of Test Example 1 had substantial anti-bacterial efficacy. The reduction factor is defined as the logarithmic reduction of bacteria after exposure with the oil-in-water emulsion according to formula (I), wherein No is the initial bacterial (or pathogen) concentration and Ne is the bacterial (or pathogen) concentration after exposure with the oil-in-water emulsion:

$$reduction factor = {log}_{10} ( {N}_{0} / {N}_{e} ) . \qquad ( I )$$

Thus, a reduction factor of 1 represents a 10-fold reduction of the bacteria and a reduction factor of 2 represents a 100-fold reduction of bacteria etc.

TABLE 1
Reduction factors after exposure to oil-in-water emulsion
with the exposure time denoted in parenthesis

| | Test | Comparative | Test | Comparative |
|---|---|---|---|---|
| | Example 1 | Example 1 | Example 1 | Example 2 |
| | (300 sec) | (300 sec) | (600 sec) | (600 sec) |
| S. aureus | 3.30 | 0.66 | 5.37 | 0.70 |
| E. coli | 5.31 | 1.09 | 5.18 | 0.51 |
| E. hirae | 4.31 | 0.7 | 1.62 | 0.85 |
| P. aeruginosa | 5.28 | 2.43 | 5.42 | 0.75 |

It can be seen from the above that the anti-bacterial efficacy of the claimed composition (Test Example 1) is greatly improved over a prior art composition containing less ethanol (Comparative Example 1) or over a composition not containing ethanol (Comparative Example 2). In addition, it can be seen that the claimed composition even shows greater anti-bacterial efficacy after a short exposure time of 300 seconds compared to a composition not containing ethanol after an exposure time of 600 seconds.

### Example 3: In Vitro Anti-Mycotic Efficacy

The oil-in-water emulsion of Example 1 was tested undiluted in accordance with DIN EN ISO 1650 against C. albicans. The exposure time was 60 seconds It was found that the oil-in-water emulsion had substantial anti-mycotic efficacy.

### Example 4: In Vitro Antiviral Efficacy Against Vacciniavirus

The oil-in-water emulsion of Example 1 was tested undiluted in accordance with DIN EN ISO 16777 on in-vitro skin against Vacciniavirus. The exposure time was 60 seconds. It was found that the oil-in-water emulsion had substantial antiviral efficacy.

### Example 5: In Vitro Antiviral Efficacy Against Bovine Coronavirus

The oil-in-water emulsion of Test Example 1 was tested in accordance with DIN EN 14476 with bovine Coronavirus. The oil-in-water emulsion of Test Example 1 resulted in a reduction factor of the amount of virus after exposure of 4.54.

### Example 6: In Vivo Antiviral Efficacy

Murine norovirus was applied to the hands of 5 patients. Then, the oil-in-water emulsion of Test Example 1 was applied on the skin of the hands of 5 patients. After an exposure time of 120 seconds, the reduction of the amount of virus was measured according to EN 17430.

TABLE 2
In-vivo reduction factor

| | Test Example 1 |
|---|---|
| Patient 1 | 1.07 |
| Patient 2 | 0.82 |
| Patient 3 | 1.50 |
| Patient 4 | 1.82 |
| Patient 5 | 1.56 |

Thus, after applying the oil-in-water emulsion of Test Example 1, a reduction of virus load can be observed.

1. An oil-in-water emulsion comprising
(a) an oil phase;
(b) an aqueous phase; and
(c) an emulsifier,
wherein the emulsion further comprises 15.0 to 30.0 wt.-% ethanol.

2. The oil-in-water emulsion according to claim 1, further comprising 1.0 to 10.0 wt.-% of cetearyl ethylhexanoate as a skin care component.

3. The oil-in-water emulsion according to claim 2, comprising the emulsifier in an amount of 1.0 to 15.0 wt.-%.

4. The oil-in-water emulsion according to claim 2, wherein the emulsifier is selected from the group of fatty alcohol esters, glycerol fatty acid esters, polyoxyethylene ethers of one or more fatty alcohols, polyglycerol ethers of fatty alcohols, polyglycerol esters of fatty acid, and mixtures thereof.

5. The oil-in-water emulsion according to claim 2, comprising the oil phase in an amount of 0.1 to 20.0 wt.-%, wherein the oil phase comprises fatty alcohols, alkylcarbonates, alkylesters, waxes, natural oils, or mixtures thereof.

6. The oil-in-water emulsion according to claim 2, further comprising a thickening agent.

7. The oil-in-water emulsion according to claim 6, wherein the thickening agent is selected from xanthan gum, polyacrylic acid, cellulose ethers, and mixtures thereof.

8. The oil-in-water emulsion according to claim 2, further comprising 0.01 to 5.0 wt.-% of an emollient.

9. The oil-in-water emulsion according to claim 8, wherein the emollient is dimethicone.

10. The oil-in-water emulsion according to claim 2, wherein the weight ratio (b)/(a) is from 2.0 to 15.0.

11. The oil-in-water emulsion according to claim 2, further comprising a vitamin.

12. The oil-in-water emulsion according to claim 1, comprising: 40.0 to 80.0 wt.-% water phase, 0.1 to 20% oil phase, 1.0 to 15.0 wt.-% of an emulsifier, 1.0 to 10.0 wt.-% of cetearyl ethylhexanoate, and 0.01 to 3 wt.-% of a thickening agent.

13. A method of preventing or treating a bacterial, mycotic, or enveloped viral disease comprising administering the oil-in-water emulsion of claim 2 to a subject or patient.

14. The method of claim 13, wherein the oil-in-water emulsion is topically applied to a portion of skin of the subject or patient.

15. The method of claim 13, wherein the oil-in-water emulsion is topically applied to a hand of the subject or patient.

16. The method of claim 13, wherein the disease is an infection with an enveloped virus belonging to the group consisting of Poxviridae, Paramyxoviridae, Filoviridae, Orthomyxoviridae, Astroviridae, and Coronaviridae.

17. The method of claim 13, wherein the disease is an infection with a bacterium belonging to the group of Staphylococcus aureus, Enterococcus hirae, Pseudomonas aeruginosa, Escherichia coli, Haemophilus influenza, and Streptococcus pneumoniae.

18. The method of claim 13, wherein the disease is an infection with a virus belonging to the family of Coronaviridae.

19. The method of claim 18, wherein the disease is an infection of Sars-COV-2.

20. A method of moisturizing and disinfecting skin of a subject or patient comprising topically applying the oil-in-water emulsion of claim 2 to a portion of the skin of the subject or patient.
